(19) United States
(12) Patent Application Publication
(10) Pub. No.: US 2017/0010266 A1
(43) Pub. Date: Jan. 12, 2017

(54) Biomarkers and Therapeutic Targets for Sarcoma

(71) Applicant: ISIS Innovation Limited, Oxford Oxfordshire (GB)

(72) Inventors: Terrence Rabbitts, Oxford Oxfordshire (GB); Jennifer Town, Oxford Oxfordshire (GB); Helio Pais, Oxford Oxfordshire (GB)

(73) Assignee: ISIS Innovation Limited, Oxford Oxfordshire (GB)

(21) Appl. No.: 15/119,466

(22) PCT Filed: Feb. 17, 2015
(86) PCT No.: PCT/GB2015/050450

§ 371 Date: Aug. 17, 2016

(30) Foreign Application Priority Data
Feb. 17, 2014 (GB) 1402771.8

(51) Int. Cl. G01N 33/574; C12N 5/0783; C12N 15/115; C12Q 1/68; C07K 16/28

(52) U.S. Cl. CPC G01N 33/57407; C12Q 1/6886; C07K 16/2803; C12N 15/115; C12N 5/0636; C12Q 2600/158; G01N 2333/70503; C07K 2317/732; C12N 2310/16; C12N 2320/30; C12N 2510/00

(57) ABSTRACT

New sarcoma cell surface targets have been identified and include LINGO-1, KCNN1 and CDH23. Specific binding agents that specifically bind to these targets include antibodies and aptamers. Methods of treating sarcoma utilize the specific binding agents. Methods of diagnosing and detecting sarcoma, including metastases and residual disease involve determining the expression of LINGO-1, KCNN1 and CDH23.

### FIELD OF THE INVENTION

The present invention relates to sarcoma. Provided are molecules and methods for detecting and treating sarcoma in a subject. Kits and systems useful in the methods are also described.

### BACKGROUND OF THE INVENTION

A sarcoma is a cancer derived from cells of mesenchymal origin. Ewing's sarcoma is an undifferentiated small blue round cell tumour that mainly appears in bone and less frequently in soft tissue. It is the second most common tumour of children and adolescents with the mean age of diagnosis being 15 years of age. Due to its high propensity to metastasize about 15-25% of patients present with metastasis at the time of diagnosis, the most common sites being lung, bone and bone marrow (Sankar, S. & Lessnick, S. L. Cancer genetics, 2011, 204, 351-365; Terrier, P., Llombart-Bosch, A. & Contesso, G. Seminars in diagnostic pathology 1996, 13, 250-257). The current standard of care treatment is multimodal treatment including systemic chemotherapy with either radiation or surgery, often with limb amputation in patients with local recurrence (Abed, R. & Grimer, R. Cancer treatment reviews 2010, 36, 342-347; Potratz, J., Dirksen, U., Jurgens, H. & Craft, A. Pediatric hematology and oncology 2012, 29, 1-11). However, despite aggressive treatment the 5-year survival rate is 60-70% for localized disease and sharply drops to a poor 10-30% if the tumour had metastasized (Sankar, S. & Lessnick, S. L. Cancer genetics, 2011, 204, 351-365; Gorlick, R., Janeway, K., Lessnick, S., Randall, R. L. & Marina, N. Pediatric blood & cancer 2013, 60, 1009-1015). There is therefore an un-met need for novel targeted therapies for Ewing's sarcoma, which overcome the main limitations of the current treatment regimens, namely the severe side effects and very limited effectiveness for metastasized disease.

Cell surface proteins are excellent target molecules in the context of antibody-based therapeutics and diagnostics. Recently, Next Generation Deep sequencing has been applied to RNA sequencing (RNA-seq) and this has emerged as a new method for the comprehensive analysis of whole cellular transcriptomes and is a powerful surrogate tool for the analysis of the cell surface proteins (herein termed the surfaceome) (da Cunha, J. P. et al. Proceedings of the National Academy of Sciences of the United States of America 2009, 106, 16752-16757; Wang, Z., Gerstein, M. & Snyder, M. Nature reviews. Genetics 2009, 10, 57-63). Cellular RNA is converted into a library of cDNA, which is then sequenced in a high-throughput manner.

### DESCRIPTION OF THE INVENTION

The present invention is based upon the identification by the present inventors that the cell surface protein LINGO-1 is up-regulated in Ewing's Sarcoma.

The present inventors carried out RNA-seq deep sequencing on three different human Ewing's Sarcomas (EWS) and compared these data with two mesenchymal stem cell (MSC) lines (MSC is thought to be the cell of origin of Ewing's sarcoma). Using an improved version of the original surfaceome database (da Cunha, J. P. et al. Proceedings of the National Academy of Sciences of the United States of America 2009, 106, 16752-16757) the present inventors have found several mRNAs that encode for cell surface proteins that are up-regulated in EWS compared with the MSCs. One of these encodes LINGO-1. Examination of LINGO-1 protein expression by Western blotting confirmed the RNA findings and showed a direct correlation to mRNA level.

LINGO-1 is also known in the art by the names LRRN6, LRRN6A, FLJ14594, LERN1, MGC17422 and UNQ201. The Entrez gene number for LINGO-1 is NM_032808.5 and the UniProt accession number is Q96FE5. In healthy individuals, LINGO-1 is exclusively expressed in the central nervous system where it plays a role in the regulation of neuronal survival, axon regeneration, oligodendrocyte differentiation and myelination (Mi, S., Sandrock, A. & Miller, R. H. The international journal of biochemistry & cell biology, 2008, 40, 1971-1978; Mi, S. et al. Nature neuroscience 2005, 8, 745-751; Llorens, F. et al. Developmental neurobiology 2008, 68, 521-541). LINGO1 consists of 620 amino acids with a large extracellular domain (42-561) the structure of which has been elucidated by crystallography (Mosyak, L. et al. The Journal of biological chemistry, 2006, 281, 36378-36390). Therefore, LINGO-1 has a well-defined and accessible extracellular domain.

Further mRNAs encoding cell surface proteins that are up-regulated in EWS compared with MSCs, as discovered by the inventors, include KCNN1 and CDH23. KCNN1 is the approved symbol (HUGO Gene Nomenclature Committee) for potassium channel, calcium activated intermediate/small conductance subfamily N alpha, member 1. KCNN1 is also known in the art by the symbols/names “potassium intermediate/small conductance calcium-activated channel, subfamily N, member 1”, hSK1, KCa2.1 and “small conductance calcium-activated potassium channel 1”. The Entrez gene number for KCNN1 is 3780, the RefSeq is NM_002248 and the UniProt accession number is Q92952. CDH23 is the approved symbol (HUGO Gene Nomenclature Committee) for cadherin-related 23. CDH23 is also known in the art by the symbols/names “cadherin related 23”, “cadherin-like 23”, DFNB12, USH1D, “cadherin-related family member 23” and CDHR23. The Entrez gene number for CDH23 is 64072, the RefSeq is NM_052836 and the UniProt accession number is Q9H251.

Thus, in a first aspect the invention provides a specific binding agent that can specifically bind to LINGO-1 polypeptide and elicit an anti-tumour response. Similarly, the invention provides a specific binding agent that can specifically bind to a polypeptide selected from KCNN1 and CDH23 and elicit an anti-tumour response.

The specific binding agent may comprise, consist essentially of or consist of an antibody or an aptamer or a protein or a peptide. (as defined herein). Comprise and related terms are intended to be interpreted openly as including the defined component in a manner that retains functionality. Consist on the other hand refers to the defined component only. Consisting essentially refers to the defined component with the potential for minor additions or modifications that do not affect function.

By anti-tumour response is meant any response in a subject to which the specific binding agent is administered that results in reduced tumour size or eradication of the tumour. Prevention of growth of the tumour may be considered an anti-tumour response in some embodiments. The anti-tumour response may rely upon antagonism of LINGO-1 function/activity or of KCNN1 or CDH23 function/activity (as appropriate). The specific binding agent may therefore be considered an antagonist of LINGO-1 or of KCNN1 or CDH23 (respectively). In certain embodiments the tumour is a sarcoma. The anti-tumour response may rely upon killing of tumour cells (which express LINGO-1 or which express KCNN1 or CDH23). The anti-tumour response may be mediated by an antibody-drug conjugate (ADC). In certain embodiments the anti-tumour response relies on cell-mediated cytotoxicity. The anti-tumour response may be Antibody-Dependent Cell-Mediated Cytotoxicity (ADCC) or Aptamer-Dependent Cell-Mediated Cytotoxicity. ADCC is a cell-mediated immunity mechanism whereby an effector cell of the immune system actively lyses a target cell, whose surface antigens have been bound by specific antibodies. ADCC may involve activation of NK cells by antibodies. NK cells express the Fc receptor CD16, which recognizes and binds to the Fc region of an antibody that has bound to the surface of the target cell. Once the Fc receptor binds to the Fc region of the antibody the NK cell releases cytokines. Aptamer-Dependent Cell-Mediated Cytotoxicity is an analogous mechanism using an aptamer rather than an antibody (Boltz A, et al. (2011) The Journal of biological chemistry 286: 21896-21905). The aptamer may be bi-specific, preferably binding specifically to LINGO-1, or to KCNN1 or CDH23 in some embodiments, as well as an Fc receptor, for example the CD16 receptor (Boltz et al, 2011 The Journal of biological chemistry 286: 21896-21905).

The skilled person is well able to identify a specific binding agent that can elicit an anti-tumour response. Suitable methods for determining the efficacy of an antibody or an aptamer in eliciting ADCC include chromium-51 [Cr⁵¹], europium [Eu] and sulphur-35 [S³⁵] release assays. In general, a target cell line containing an intracellular label (e.g. Cr⁵¹, Eu or S³⁵) and expressing a particular surface antigen is incubated with an antibody specific for that antigen. After washing, effector cells expressing Fc receptor CD16 are co-incubated with the antibody-bound target cells. Measurement of the release of intracellular label using a scintillation counter or spectrophotometry allows target cell lysis to be determined. Further methods that rely on determining target cell lysis include the Calcein-AM (calcein-acetoxymethyl) assay. The dye is retained in viable cells, but released as the fluorescent compound, calcein, by apoptotic cells. Additionally, target cell lysis may be measured by the dissociation-enhanced fluorescent immunoassay (DELFIA), which involves loading target cells with bis(acetoxymethyl) 2,2′:6′,2″-terpyridine-6,6″-dicarboxylate (BATDA). Inside the cell membrane, ester bonds hydrolyse to form 2,2′:6′2″-terpyridine-6,6″-dicarboxylic acid (TDA), a non-cell permeable hydrophilic ligand. Upon cell lysis the released TDA ligand now in solution binds to europium to form the highly fluorescent and stable chelate, EuTDA. The fluorescent signal is measured via time-resolved fluorescence (TRF), and directly correlates with the amount of target cell lysis. An additional method to determine the efficacy of an antibody or an aptamer to elicit ADCC is measurement of activation of the NFAT (nuclear factor of activated T-cells) pathway in the effector cell. The use of engineered Jurkat cells stably expressing the CD16 receptor and an NFAT response element driving expression of firefly luciferase as effector cells allows the quantification of ADCC by measuring luciferase activity as a luminescent readout. (Parekh et al, 2012 Development and validation of an antibody-dependent cell-mediated cytotoxicity-reporter gene assay. mAbs 4: 310-318)

According to a further aspect of the invention there is provided a specific binding agent that can specifically bind to LINGO-1 polypeptide operably connected to:
(i) a cytotoxic agent or prodrug thereof,
(ii) an agent capable of activating a prodrug that when activated is a cytotoxic agent; or
(iii) a delivery agent containing a cytotoxic agent or prodrug thereof.

Similarly, the invention also provides a specific binding agent that can specifically bind to KCNN1 or CDH23 polypeptide operably connected to:
(i) a cytotoxic agent or prodrug thereof,
(ii) an agent capable of activating a prodrug that when activated is a cytotoxic agent; or
(iii) a delivery agent containing a cytotoxic agent or prodrug thereof.

In yet a further aspect, the present invention relates to a pharmaceutical composition comprising a specific binding agent according to the present invention and a pharmaceutically acceptable carrier or excipient.

The invention also relates to a kit for targeting LINGO-1 expressing cells comprising:
a. a specific binding agent that can specifically bind to LINGO-1 polypeptide; and
b. one or more of:
   i. a cytotoxic agent or prodrug thereof,
   ii. an agent capable of activating a prodrug that when activated is a cytotoxic agent;or
   iii. a delivery agent containing a cytotoxic agent or prodrug thereof.

The invention also relates to a kit for targeting KCNN1 or CDH23 expressing cells comprising:
a. a specific binding agent that can specifically bind to KCNN1 or CDH23 polypeptide; and
b. one or more of:
   iv. a cytotoxic agent or prodrug thereof,
   v. an agent capable of activating a prodrug that when activated is a cytotoxic agent;or
   vi. a delivery agent containing a cytotoxic agent or prodrug thereof.

Components a and b of the kits are operably connectable to one another. The kits may therefore further comprise means for operably connecting components a and b.

In a further aspect, the present invention relates to a specific binding agent or pharmaceutical composition of the present invention for use as a medicament. Similarly, the invention provides a specific binding agent or pharmaceutical composition of the present invention for use in the manufacture of a medicament.

In yet a further aspect, the present invention relates to a specific binding agent or pharmaceutical composition of the present invention for use in a method of treating sarcoma (such as Ewing's sarcoma). Similarly, the invention provides a specific binding agent or pharmaceutical composition of the present invention for use in the manufacture of a medicament for treating sarcoma.

The invention also relates to a specific binding agent that can specifically bind to LINGO-1 polypeptide for use in a method of treating sarcoma. The invention also relates to a specific binding agent that can specifically bind to KCNN1 or CDH23 polypeptide for use in a method of treating sarcoma.

According to a further aspect of the invention there is provided a method for treating sarcoma in a subject comprising inhibiting LINGO-1 expression or LINGO-1 protein activity. In certain embodiments the method comprises administering to a subject a specific binding agent that can specifically bind to LINGO-1 polypeptide.

Similarly, there is provided a method for treating sarcoma in a subject comprising inhibiting KCNN1 or CDH23 expression or KCNN1 or CDH23 protein activity. In certain embodiments the method comprises administering to a subject a specific binding agent that can specifically bind to KCNN1 or CDH23 polypeptide.

In a further aspect, the present invention relates to a method for treating sarcoma in a subject comprising administering to a subject a specific binding agent or pharmaceutical composition of the present invention.

The present inventors have found that an increased level of LINGO-1 expression is associated with sarcoma. They have also found that an increased level of KCNN1 or CDH23 expression is associated with sarcoma.

Therefore, in yet a further aspect, the present invention relates to a method for detecting and/or diagnosing sarcoma in a subject comprising:

determining the expression level of LINGO-1 in a sample from the subject wherein the determined expression level is used to detect and/or diagnose sarcoma in the subject.

Similarly, the invention provides a method for detecting and/or diagnosing sarcoma in a subject comprising:

determining the expression level of KCNN1 or CDH23 in a sample from the subject wherein the determined expression level is used to detect and/or diagnose sarcoma in the subject.

The invention also relates to use of a specific binding agent that binds specifically to LINGO-1 for detecting and/or diagnosing sarcoma in a subject. Similarly, the invention also relates to use of a specific binding agent that binds specifically to KCNN1 or CDH23 for detecting and/or diagnosing sarcoma in a subject.

According to a further aspect of the invention there is provided a method for detecting and/or diagnosing sarcoma in a subject comprising:

determining the expression level of LINGO-1 in a sample from the subject in order to identify the presence or absence of cells characteristic of sarcoma wherein the determined presence or absence of the cells is used to detect and/or diagnose sarcoma in the subject. Similarly, the invention provides a method for detecting and/or diagnosing sarcoma in a subject comprising:

determining the expression level of KCNN1 or CDH23 in a sample from the subject in order to identify the presence or absence of cells characteristic of sarcoma wherein the determined presence or absence of the cells is used to detect and/or diagnose sarcoma in the subject.

In a further aspect the invention provides a method for detecting metastases in a subject with sarcoma comprising:

determining the expression level of LINGO-1 in a sample from the subject wherein the determined expression level is used to detect metastases.

Similarly, the invention provides a method for detecting metastases in a subject with sarcoma comprising:

determining the expression level of KCNN1 or CDH23 in a sample from the subject wherein the determined expression level is used to detect metastases.

In yet a further aspect, the present invention relates to a method for identifying residual disease in a subject following treatment for sarcoma comprising:

determining the expression level of LINGO-1 in a sample from the subject wherein the determined expression level is used to identify residual disease in the subject.

Similarly, the invention relates to a method for identifying residual disease in a subject following treatment for sarcoma comprising:

determining the expression level of KCNN1 or CDH23 in a sample from the subject wherein the determined expression level is used to identify residual disease in the subject.

The invention also relates to a method for detecting and/or diagnosing sarcoma in a subject comprising:
a. obtaining a sample from the subject
b. applying a specific binding agent that can specifically bind to LINGO-1 polypeptide to the sample from the subject
c. applying a detection agent that detects the a specific binding agentrLINGO-1 polypeptide complex
d. using the detection agent to determine the level of LINGO-1 polypeptide wherein the determined level of the LINGO-1 polypeptide is used to detect and/or diagnose sarcoma in the subject.

Similarly, the invention also relates to a method for detecting and/or diagnosing sarcoma in a subject comprising:
a. obtaining a sample from the subject
b. applying a specific binding agent that can specifically bind to KCNN1 or CDH23 polypeptide to the sample from the subject
c. applying a detection agent that detects the specific binding agent-KCNN1 or specific binding agent-CDH23 polypeptide complex
d. using the detection agent to determine the level of KCNN1 or CDH23 polypeptide wherein the determined level of the KCNN1 or CDH23 polypeptide is used to detect and/or diagnose sarcoma in the subject.

In specific embodiments (according to the various aspects) an increased expression level of LINGO-1, or of KCNN1 or CDH23, is used to detect and/or diagnose sarcoma and/or to detect metastases and/or to identify residual disease. In certain embodiments an increased expression level of LINGO-1, or of KCNN1 or CDH23, is indicative of sarcoma and/or metastases in a subject with sarcoma and/or residual disease in a subject following treatment for sarcoma.

The specific binding agent that can specifically bind to LINGO-1 polypeptide, or to the KCNN1 or CDH23 polypeptide in other aspects, may be incorporated into a chimeric antigen receptor (CAR). Chimeric antigen receptors (CARs) are proteins which graft the specificity of a binding agent such as a monoclonal antibody to the effector function of a T-cell. Typically in these embodiments, the antigen-binding fragment may comprise an scFv molecule that specifically binds to LINGO-1, or to KCNN1 or CDH23. The invention thus also provides a CAR comprising:

1. A specific binding agent that can specifically bind to LINGO-1 polypeptide (antigen binding domain)

2. A spacer or hinge domain to elevate the antigen binding domain from the cell surface

3. A trans-membrane domain (TM) to anchor the CAR within the T cell membrane

4. An endodomain which transmits signals within the T cell.

The invention thus also provides a CAR comprising:

1. A specific binding agent that can specifically bind to KCNN1 or CDH23 polypeptide (antigen binding domain)

2. A spacer or hinge domain to elevate the antigen binding domain from the cell surface

3. A trans-membrane domain (TM) to anchor the CAR within the T cell membrane

4. An endodomain which transmits signals within the T cell.

The endodomains have evolved from the first generation domains which transmitted an ITAM signal alone, to second and third generation endodomains which transmit a further one or two co-stimulatory signals in cis. The endodomain thus typically contains an activating domain such as a CD3-zeta endodomain. The CAR may additionally or alternatively comprise co-stimulatory domains such as CD28, 41 BB and/or OX40 domains.

The invention also relates to nucleic acid molecules encoding the CARs, together with vectors and host cells. The invention also relates to a (heterologous) T cell comprising a CAR of the invention, together with pharmaceutical compositions comprising such CARs together with a suitable carrier or excipient. The invention also relates to autologous T cell therapies for sarcoma incorporating the T cells, compositions and CARs of the invention (and corresponding medical uses).

According to all aspects of the invention the specific binding agent may be an antibody or an aptamer or a protein or a peptide. Preferably, the specific binding agent is an antibody or an aptamer.

According to all aspects of the invention the antibody may be of monoclonal or polyclonal origin. Typically, the antibody is an IgG immunoglobulin isotype. Antigen-binding fragments and antibody derivatives may also be utilised, to include without limitation Fab fragments, ScFv, single domain antibodies, nanoantibodies, heavy chain antibodies, chimeric antibody fusions etc. which retain antigen-specific binding function and these are included in the definition of “antibody”. Methods for generating specific antibodies are known to those skilled in the art. Antibodies may be of human or non-human origin (e.g. rodent, such as rat or mouse) and be humanized etc. according to known techniques (Jones et al., Nature (1986) May 29-Jun. 4;321(6069):522-5; Roguska et al., Protein Engineering, 1996, 9(10):895-904; and Studnicka et al., Humanizing Mouse Antibody Frameworks While Preserving 3—D Structure. Protein Engineering, 1994, Vol.7, pg 805).

In all aspects of the invention the aptamer may be a nucleic acid molecule, to include natural nucleotides and derivatives/analogues and non-natural nucleotides, or a peptide molecule, to include natural amino acids and derivatives/analogues and non-natural amino acids bonded with standard or non-standard peptide bonds, or one or more operably connected nucleic acid molecules and/or peptide molecules. In specific embodiments the aptamer is a DNA, RNA or XNA aptamer. Nucleic acid aptamer selection can be made using methods known to those skilled in the art, for example using in vitro selection or SELEX. In certain embodiments the aptamer is a peptide aptamer comprising a variable peptide domain, attached at both ends to a protein scaffold. The variable peptide domain may be up to 30 amino acids long, preferably 5 to 25 amino acids long, more preferably 10 to 20 amino acids long. The scaffold may be any protein which has appropriate solubility and compacity properties, for example the bacterial protein Thioredoxin-A, Peptide aptamer selection can be made using methods known to those skilled in the art, for example using the yeast two-hybrid system.

By “specifically bind” it is meant that an antibody binds to an epitope via its antigen binding domain, and that the binding entails some complementarity between the antigen binding domain and the epitope. The term is well known in the art. Accordingly, an antibody specifically binds to an epitope when it binds to that epitope, via its antigen binding domain more readily than it would bind to a random, unrelated epitope. Likewise, a specific binding agent such as an aptamer, protein or peptide specifically binds to a target molecule when it binds to that molecule more readily than it would to a random, unrelated molecule. The dissociation constant or Kd may be less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁵ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×1⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

A specific binding agent that can specifically bind to LINGO-1 polypeptide, or to KCNN1 or CDH23 polypeptide in other aspects, is considered to be “operably connected” to:
(i) a cytotoxic agent or prodrug thereof,
(ii) an agent capable of activating a prodrug that when activated is a cytotoxic agent; or
(iii) a delivery agent containing a cytotoxic agent or prodrug thereof

if the cytotoxic agent or prodrug (i), agent capable of activating a prodrug (ii) or delivery agent (iii) is able to function such that the LINGO-1 expressing cell, or KCNN1 or CDH23 expressing cell in other aspects, to which the specific binding agent is bound can be killed (as a consequence of/following binding). The specific binding agent that can specifically bind to LINGO-1, or to KCNN1 or CDH23 in other aspects, may be recombinantly fused or chemically conjugated (including covalent and non-covalent conjugations) to the cytotoxic agent or prodrug (i), agent capable of activating a prodrug (ii) or delivery agent (iii). Fusion proteins can be prepared using methods that are well known in the art. The position at which the fusion is made may be selected using methods known to the skilled person to optimize the secretion or binding characteristics of the fusion protein. DNA encoding the fusion protein is then transfected into a host cell for expression. Conjugates may also be assembled using a variety of techniques well known in the art depending on the selected agent to be conjugated. A specific binding agent that can specifically bind to LINGO-1 polypeptide, or to KCNN1 or CDH23 polypeptide in other aspects, can be labeled or conjugated either before or after purification, when purification is performed. Techniques for conjugating various moieties to specific binding agent are well known, see for example, Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

A linker may be used to connect the specific binding agent that can specifically bind to LINGO-1 polypeptide, or to KCNN1 or CDH23 polypeptide in other aspects, to the cytotoxic agent or prodrug (i), agent capable of activating a prodrug (ii) or delivery agent (iii). In specific embodiments the linker is based on a chemical motif including disulphde, hydrazone, peptide or thioether. The linker may also be a peptide linker and may, in some embodiments, be incorporated into a fusion protein with the specific binding agent that can specifically bind to LINGO-1 polypeptide, or to KCNN1 or CDH23 polypeptide in other aspects. The linker may be cleavable or non-cleavable.

In certain embodiments the cytotoxic agent is a chemotherapeutic drug, an RNA molecule (optionally a shRNA, siRNA or antisense RNA), a RNA expression vector, an antibody, an aptamer or a dominant negative protein fragment. The chemotherapeutic drug may be a platinum based agent and/or a taxane. In specific embodiments the platinum based agent is selected from cisplatin, carboplatin and oxaliplatin. The taxane may be paclitaxel, cabazitaxel or docetaxel. The cytotoxic agent may also be a vinca alkaloid, such as vinorelbine or vinblastine. The cytotoxic agent may be a topoisomerase inhibitor such as etoposide or an anthracycline (antibiotic) such as doxorubicin. The cytotoxic agent may be an alkylating agent such as estramustine. In specific embodiments the antibody, aptamer or dominant negative protein fragment is capable of reducing the expression and/or inhibiting the activity of the EWS-FLI1 fusion protein.

The RNA expression vector encodes an RNA molecule and may encode a shRNA and/or an siRNA and/or antisense RNA. In specific embodiments the shRNA and/or siRNA and/or antisense RNA is capable of reducing expression of the EWS-FLI1 fusion protein. The EWS-FLI1 fusion protein resulting from a t(11;22)(q24;q12) translocation is found in 85-90% of Ewing's sarcoma. In rare cases this translocation and concomitant expression of EWS-FLI1 has been reported to occur in neuroblastoma and rhabdomyosarcoma. For EWS-FLI1 Type 1 the Entrez gene number is AF327066.1 and the UniProt accession number is Q9BZD1. EWS is a member of the FET family of proteins, which are involved in transcription and RNA processing. FLI1 belongs to the ETS family of transcription factors. In frame fusion of the N-terminal transactivation domain of EWS with the C-terminal DNA binding domain of FLI1 yields a highly expressed, non-physiologic, oncogenic transcription factor, which exerts activating and repressive effects on gene expression. EWS-FLI1 is essential for oncogenic transformation and induces immunohistologic features of Ewing's sarcoma in several immortalized cell lines (Thorner P, Squire J, Chilton-MacNeil S, Marrano P, Bayani J, Malkin D, Greenberg M, Lorenzana A, Zielenska M. Am J Pathol. 1996 Apr;148(4):1125-38; Burchill S A, Wheeldon J, Cullinane C, Lewis I J. Eur J Cancer. 1997 Feb;33(2):239-43; Nicolà Riggi, Mario-Luca Suvà, Domizio Suva, et al. Cancer Res 2008;68:2176-2185). shRNA vectors used for down-regulation of the EWS-FLI1 fusion protein are described in Smith R, Owen L A, Trem D J, Wong J S, Whangbo J S, Golub T R, Lessnick S L (2006) Cancer cell 9: 405-416, incorporated herein by reference. One shRNA vector used for down-regulation of the EWS-FLI1 fusion protein is pSRP-EF3′-2.

The shRNA sequence within pSRP-EF3′-2 (targeting EWS-FLI1) is

```
(SEQ ID NO: 1)
                            sense
GATCCCCATAGAGGTGGGAAGCTTATTTCAAGA
              anti-sense
GAATAAGCTTCCCACCTCTATTTTTTGGAACTC
GAGGT
```

Target sequences are shown in bold. This example shRNA binds in the 3′ untranslated region of FLI1. Any suitable portion of the EWS-FLI1 sequence may be targeted.

In further embodiments, in addition or as an alternative to reducing expression of the EWS-FLI1 fusion protein, the shRNA and/or siRNA and/or antisense RNA is capable of reducing expression of one or more of the following fusion proteins: EWS-ERG, EWS-FEV, FUS-ERG, FUS-FEV, EWS-ETV; and/or STK10 (serine threonine kinase 10), TNK2 (tyrosine kinase, nonreceptor,2), PLK1 (polo like kinase 1) (Arora et al, 2010 Molecular cancer 9: 218) and HSP90 (heat shock protein 90) (Ambati et al, 2013 Pre-clinical efficacy of PU-H71, a novel HSP90 inhibitor, alone and in combination with bortezomib in Ewing sarcoma. Molecular oncology).

In certain embodiments the RNA expression vector encodes an antibody, dominant negative protein fragment or aptamer capable of reducing expression of and/or inhibiting the activity of the EWS-FLI1 fusion protein.

A pro-drug of a cytotoxic agent is such an agent that is in an inactive or less than fully active form and can be converted to its active form. In certain embodiments the agent capable of activating a pro-drug is an enzyme.

The agent capable of activating the pro-drug, typically an enzyme, is operably connected to the specific binding agent that can specifically bind to LINGO-1 polypeptide, or to KCNN1 or CDH23 polypeptide in other aspects, in a manner such that the agent/enzyme is delivered to the target location by virtue of its association with the specific binding agent that can specifically bind to LINGO-1 polypeptide, or to KCNN1 or CDH23 polypeptide in other aspects. As already discussed, the agent may be covalently linked to the specific binding agent that can specifically bind to LINGO-1 polypeptide, or to KCNN1 or CDH23 polypeptide in other aspects. The agent/enzyme acts to convert a pro-drug to an active drug at the target location (i.e at the tumor where the cells express LINGO-1, or at the tumor where the cells express KCNN1 or CDH23 in other aspects). The enzyme is preferably one that is not naturally present in the subject to prevent undesired activity and side effects in the subject. The enzyme modifies a pro-drug molecule that is non-toxic until acted upon by the enzyme to release an active drug. This may be a short lived active drug in some embodiments to minimize off-target toxicity. Thus, the localisation of enzyme activity ensures that the pro-drug is activated/cleaved only in the immediate vicinity of the target location.

Any suitable enzyme may be employed that permits conversion of inactive pro-drug to active drug at the target location. The enzyme may be a cleavage enzyme such as a protease. Suitable enzymes include carboxypeptidase G2, alkaline phosphatase, beta-glucoronidase, penicillin-V-amidase, beta-lactamase, beta-glucosidase and nitroreductase. Potentially useful enzyme/pro-drug combinations, which may be operably connected to the specific binding agent that can specifically bind to LINGO-1 polypeptide, or to KCNN1 or CDH23 polypeptide in other aspects, are listed in table A.

TABLE A
Enzymes, pro-drugs and corresponding drugs which may
be useful in the invention

| Enzyme | Prodrug | Drug |
|---|---|---|
| Carboxypeptidase G2 | Benzoic acid mustard | Benzoic acid mustards |
| | glutamates | |
| Cytosine deaminase | 5-fluorocytosine | 5-fluorouracil |
| Carboxypeptidase A | Methotrexate-alanine | Methotrexate |
| Alpha-galactosidase | N-[4-(α-D- | Daunorubicin |
| | galactopyranosyl)- | |
| | benzyloxycarbonyl]- | |
| | daunorubicin | |
| Beta-glucosidase | Amygdalin | Cyanide |
| Beta-lactamase | Vinca-cephalosporin | 4- |
| | | desacetylvinblastine- |
| | | 3-carboxyhydrazide |
| | Phenylenediamine | Phenylenediamine |
| | mustard- | mustard |
| | cephalosporin | |
| | Nitrogen-mustard- | Nitrogen-mustards |
| | cephalosporin | |
| Alkaline phosphatase | Phenolmustard phosphate | Phenolmustard |
| | Doxorubicin phosphate | Doxorubicin |
| | Mitomycin phosphate | Mitomycin alcohol |
| | Etopside phosphate | Etopside |
| Penicillin amidase | Palytoxin-4- | Palytoxin |
| | hydroxyphenyl- | |
| | acetamide | |
| | Doxorubicin- | Doxorubicin |
| | phenoxyacetamide | |
| | Melphalan- | Melphalan |
| | phenoxyacetamide | |
| Nitroreductase | CB1954 | 5-(aziridine-1-yl)-4- |
| | | hydroxylamino-2- |
| | | nitrobenzamide |

In certain embodiments the delivery agent is a polymeric micelle, a liposome, a lipoprotein-based drug carrier, a nano-particle drug carrier, a lipid nanoparticle or a dendrimer. In specific embodiments the delivery agent is a liposome. The skilled person is well aware of methods by which liposomes may be operably connected to specific binding agents. The liposome may be polyethylene glycol (PEG) coated. Such a liposome may be connected to an antibody by reaction of the hydrazine group on PEG with the oxidized carbohydrate groups of the oligosaccharide portion of the antibody. Alternatively, liposomes with PDP (pyridyldithioproprionate) modified lipid may be conjugated to maleimide-modified specific binging agents. A further method of conjugation is via biotin/avidin binding using a biotinylated liposome and a specific binding agent which is covalently bound to avidin. A thiol-modified specific binding agent may be connected to a maleimide-activated liposome. Crosslinking agents, such as EDC and SPDP, S-acetylthioglycolic acid N-hydroxysuccinimide ester (SATA) and 4-(p-maleimidophenyl)butyricacid N-hydroxy-succinimide ester (SMPB) may be used to connect liposomes to specific binding agents. Specific binding agents which have been activated by these crosslinkers can react with activated lipids in liposome bilayers. For example, EDC can be used in conjunction with NHS to activate acidic functions on liposomes, which may then be conjugated to the amino groups of antibodies, prorteins, peptides or peptide aptamers (Ansell) et al, 2000 Antibody conjugation methods for active targeting of liposomes. Methods in molecular medicine 25: 51-68; Sofou & Sgouros, (2008) Antibody-targeted liposomes in cancer therapy and imaging. Expert opinion on drug delivery 5: 189-204).

The pharmaceutical compositions of the present invention may be formulated with any suitable carrier or excipient known in the art. Furthermore, the pharmaceutical compositions may be formulated into any suitable form. Examples known in the art include nanoparticles, ampoules, capsules, creams, elixirs, emulsions, microemulsions, fluids, drops, injections, solutions, lotions, sprays, powders, suspensions, syrups, tablets, tinctures or ointments.

The specific binding agent or pharmaceutical composition of the present invention may be administered by any suitable route. They may be administered systemically. Examples known in the art include intradermal, subcutaneous, intramuscular, intravenous, intraosseous, and intraperitoneal infusion or injection, oral or sublingual administration and inhalation. Similarly, the therapeutically effective dose will vary according to the severity of the disease and other patient-specific factors, such as height, age and weight of the patient. The appropriate dose can be readily determined by those of skill in the art.

In specific embodiments the specific binding agent cannot cross the blood brain barrier. This is potentially advantageous as the normal expression of LINGO-1 is believed to be exclusively in the central nervous system (CNS). By cannot cross is meant to exclude a significant and unintended effect of the specific binding agent on LINGO-1 expressed in the CNS. In some embodiments, the specific binding agent may be present in the CNS following administration at no more than approximately 0.1%, 0.05% or 0.01% of the level measured in blood. The exposure of the specific binding agent to the central nervous system may be limited by use of a molecule that is of a sufficiently large size that it does not transfuse efficiently across the blood brain barrier. For example, antibodies may be sufficiently large to prevent crossing the blood brain barrier. However, in certain embodiments the specific binding agent is connected to a further molecule in order to increase its size. The further molecule may be a carrier protein, in particular a non-immunogenic protein, in some embodiments. The specific binding agent may be pegylated. In some embodiments, the polarity of the specific binding agent may be increased as the high electrical resistance of brain capillaries forms a barrier against polar substances, while lipophilic substances are generally more permissive. Similar considerations may apply for targeting KCNN1 or CDH23.

According to all aspects of the invention the sarcoma may be a sarcoma that expresses the EWS-FLI1 fusion protein. In certain embodiments the sarcoma is Ewing's sarcoma, neuroblastoma and/or rhabdomyosarcoma. In specific embodiments the sarcoma is Ewing's sarcoma.

LINGO-1 is not only a useful therapeutic target but also a useful diagnostic biomarker. The determined expression level of LINGO-1 in a sample from a subject can be used to detect and/or diagnose sarcoma in the subject. Likewise, KCNN1 and CDH23 are not only useful therapeutic targets but also useful diagnostic biomarkers. The determined expression level of KCNN1 or CDH23 in a sample from a subject can be used to detect and/or diagnose sarcoma in the subject.

In certain embodiments the expression level of LINGO-1, or of KCNN1 or CDH23, in a sample from a subject is compared to a reference value or to the expression level in one or more control samples or to the expression level in one or more control cells in the same sample. The control cells may be normal (e.g. cells characterised by an independent method as non-sarcoma) cells. The one or more control samples may consist of non-sarcoma cells or may include a mixture of sarcoma cells and non-sarcoma cells. One or more positive control samples consisting of sarcoma cells may be used and/or one or more negative control samples consisting of non-sarcoma cells.

The reference value may be a threshold level of expression of LINGO-1, or of KCNN1 or CDH23, set by determining the level or levels in a range of samples from subjects with and without sarcoma. Suitable methods for setting a threshold are well known to those skilled in the art. The threshold may be mathematically derived from a training set of patient data. The score threshold thus separates the test samples according to presence or absence of the particular condition. The interpretation of this quantity, i.e. the cut-off threshold may be derived in a development or training phase from a set of patients with known outcome. The threshold may therefore be fixed prior to performance of the claimed methods from training data by methods known to those skilled in the art.

Methods for determining the expression levels of LINGO-1, or of KCNN1 or CDH23, are described in greater detail herein. Typically, the methods may involve contacting a sample obtained from a subject with a detection agent, such as primers/probes/antibodies (as discussed in detail herein) specific for LINGO-1, or for KCNN1 or CDH23, and detecting expression products by virtue of the specific interaction of the detection agent with the LINGO-1,or KCNN1 or CDH23 (respectively), expression products.

According to all aspects of the invention the expression level of LINGO-1, or of KCNN1 or CDH23, may be measured by any suitable method. In certain embodiments the expression level is determined at the level of protein, RNA or epigenetic modification. The epigenetic modification may be DNA methylation.

The expression level may be determined by immunohistochemistry. By immunohistochemistry is meant the detection of proteins in cells of a tissue sample by using a binding reagent such as an antibody, or aptamer that binds specifically to the protein.

In certain embodiments the expression level is determined using an antibody or aptamer (as defined herein) conjugated to a label. By label is meant a component that permits detection, directly or indirectly. For example, the label may be an enzyme, optionally a peroxidase, or a fluorophore.

A label is an example of a detection agent. By detection agent is meant an agent that may be used to assist in the detection of the antibody, or aptamer-protein complex. Where the antibody, or aptamer is conjugated to an enzyme the detection agent may be comprise a chemical composition such that the enzyme catalyses a chemical reaction to produce a detectable product. The products of reactions catalyzed by appropriate enzymes can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers. In certain embodiments the detection agent may comprise a secondary antibody. The expression level is then determined using an unlabeled primary antibody that binds to the target protein and a secondary antibody conjugated to a label, wherein the secondary antibody binds to the primary antibody.

Additional techniques for determining expression level at the level of protein include, for example, Western blot, immunoprecipitation, immunocytochemistry, mass spectrometry, ELISA and others (see ImmunoAssay: A Practical Guide, edited by Brian Law, published by Taylor & Francis, Ltd., 2005 edition). To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies.

Measuring mRNA in a biological sample may be used as a surrogate for detection of the level of the corresponding protein in the biological sample. Thus, the expression level of LINGO-1 can also be determined at the level of RNA.

Accordingly, in specific embodiments the expression level is determined by microarray, northern blotting, or nucleic acid amplification. Nucleic acid amplification includes PCR and all variants thereof such as real-time and end point methods and qPCR. Other nucleic acid amplification techniques are well known in the art, and include methods such as NASBA, 3SR and Transcription Mediated Amplification (TMA). Other suitable amplification methods include the ligase chain reaction (LCR), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995), invader technology, strand displacement technology, and nick displacement amplification (WO 2004/067726). This list is not intended to be exhaustive; any nucleic acid amplification technique may be used provided the appropriate nucleic acid product is specifically amplified. Design of suitable primers and/or probes is within the capability of one skilled in the art. Various primer design tools are freely available to assist in this process such as the NCBI Primer-BLAST tool. Primers and/or probes may be at least 15, 16, 17, 18, 19, 20, 21, 22, 23, 24 or 25 (or more) nucleotides in length. mRNA expression levels may be measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004. Expression levels may also be measured using next generation sequencing (NGS) methods, such as RNA-seq.

The methods described herein may further comprise extracting total nucleic acid or RNA from the sample. Suitable methods are known in the art and include use of commercially available kits such as Rneasy and GeneJET RNA purification kit.

In certain embodiments the methods may further comprise obtaining the sample from the subject. Typically the methods are in vitro methods performed on an isolated sample.

According to all aspects of the invention samples may be of any suitable form. The sample may comprise, consist essentially of or consist of bone and/or soft tissue.

The tissue sample may be obtained by any suitable technique. Examples include a biopsy procedure, optionally a fine needle aspirate biopsy procedure. Body fluid samples may also be utilised. Suitable sample types include blood, to encompass peripheral blood, whole blood, serum and plasma samples.

In certain embodiments detecting and/or diagnosing sarcoma in a subject comprises detecting residual disease following treatment. Thus, the method may be used to confirm that a therapeutic intervention has been successful in removing the tumour, for example following surgery or a treatment according to the invention. The methods of the invention may also be useful to monitor the treatment of an individual. For example, blood samples may be taken in conjunction with the treatment regime to determine whether LINGO-1 levels, or KCNN1 or CDH23 levels, in the sample are decreasing, indicating a successful treatment. Such monitoring may include monitoring of a treatment according to the invention.

The invention also relates to a system or device for performing a method as described herein. In a further aspect, the present invention relates to a system or test kit for detecting and/or diagnosing sarcoma in a subject comprising:
(a) one or more testing devices for determining the expression level of LINGO-1 in a sample from the subject
(b) a processor; and
(c) a storage medium comprising a computer application that, when executed by the processor, is configured to:
   (i) access and/or calculate the determined expression level of LINGO-1 in the sample on the one or more testing devices
   (ii) calculate whether there is an increased or decreased level of LINGO-1 in the sample; and
   (iii) output from the processor the detection and/or diagnosis of the sarcoma.

Similarly, the invention also relates to a system or test kit for detecting and/or diagnosing sarcoma in a subject comprising:
(a) one or more testing devices for determining the expression level of KCNN1 or CDH23 in a
sample from the subject
(b) a processor; and
(c) a storage medium comprising a computer application that, when executed by the processor, is configured to:
   (i) access and/or calculate the determined expression level of KCNN1 or CDH23 in the sample on the one or more testing devices
   (ii) calculate whether there is an increased or decreased level of KCNN1 or CDH23 in the sample; and
   (iii) output from the processor the detection and/or diagnosis of the sarcoma.

By testing device is meant a combination of components that allows the expression level of a gene to be determined. The components may include any of those described above with respect to the methods for determining expression level at the level of protein, RNA or epigenetic modification. For example the components may be antibodies, primers, detection agents and so on. Components may also include one or more of the following: microscopes, microscope slides, x-ray film, radioactivity counters, scintillation counters, spectrophotometers, colorimeters, fluorometers, luminometers, and densitometers.

In certain embodiments the system or test kit further comprises a display for the output from the processor.

The invention also relates to a computer application or storage medium comprising a computer application as defined above.

In certain example embodiments, provided is a computer-implemented method, system, and a computer program product for detecting and/or diagnosing sarcoma in a subject, in accordance with the methods described herein. For example, the computer program product may comprise a non-transitory computer-readable storage device having computer-readable program instructions embodied thereon that, when executed by a computer, cause the computer to detect and/or diagnose sarcoma in a subject as described herein. For example, the computer executable instructions may cause the computer to:
(i) access and/or calculate the determined expression level of LINGO-1, or of KCNN1 or CDH23, in a sample on one or more testing devices;
(ii) calculate whether there is an increased or decreased level of LINGO-1, or of KCNN1 or CDH23, in the sample; and,
(iii) provide an output regarding the detection and/or diagnosis of sarcoma.

In certain example embodiments, the computer-implemented method, system, and computer program product may be embodied in a computer application, for example, that operates and executes on a computing machine and a module. When executed, the application may detect and/or diagnose sarcoma in a subject, in accordance with the example embodiments described herein.

As used herein, the computing machine may correspond to any computers, servers, embedded systems, or computing systems. The module may comprise one or more hardware or software elements configured to facilitate the computing machine in performing the various methods and processing functions presented herein. The computing machine may include various internal or attached components such as a processor, system bus, system memory, storage media, input/output interface, and a network interface for communicating with a network, for example. The computing machine may be implemented as a conventional computer system, an embedded controller, a laptop, a server, a customized machine, any other hardware platform, such as a laboratory computer or device, for example, or any combination thereof. The computing machine may be a distributed system configured to function using multiple computing machines interconnected via a data network or bus system, for example.

The processor may be configured to execute code or instructions to perform the operations and functionality described herein, manage request flow and address mappings, and to perform calculations and generate commands. The processor may be configured to monitor and control the operation of the components in the computing machine. The processor may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor may be a single processing unit, multiple processing units, a single processing core, multiple processing cores, special purpose processing cores, co-processors, or any combination thereof. According to certain example embodiments, the processor, along with other components of the computing machine, may be a virtualized computing machine executing within one or more other computing machines.

The system memory may include non-volatile memories such as read-only memory (“ROM”), programmable read-only memory (“PROM”), erasable programmable read-only memory (“EPROM”), flash memory, or any other device capable of storing program instructions or data with or without applied power. The system memory may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory. The system memory may be implemented using a single memory module or multiple memory modules. While the system memory may be part of the computing machine, one skilled in the art will recognize that the system memory may be separate from the computing machine without departing from the scope of the subject technology. It should also be appreciated that the system memory may include, or operate in conjunction with, a non-volatile storage device such as the storage media. The storage media may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media may store one or more operating systems, application programs and program modules such as module, data, or any other information. The storage media may be part of, or connected to, the computing machine. The storage media may also be part of one or more other computing machines that are in communication with the computing machine, such as servers, database servers, cloud storage, network attached storage, and so forth.

The module may comprise one or more hardware or software elements configured to facilitate the computing machine with performing the various methods and processing functions presented herein. The module may include one or more sequences of instructions stored as software or firmware in association with the system memory, the storage media, or both. The storage media may therefore represent examples of machine or computer readable media on which instructions or code may be stored for execution by the processor. Machine or computer readable media may generally refer to any medium or media used to provide instructions to the processor. Such machine or computer readable media associated with the module may comprise a computer software product. It should be appreciated that a computer software product comprising the module may also be associated with one or more processes or methods for delivering the module to the computing machine via a network, any signal-bearing medium, or any other communication or delivery technology. The module may also comprise hardware circuits or information for configuring hardware circuits such as microcode or configuration information for an FPGA or other PLD.

The input/output (“I/O”) interface may be configured to couple to one or more external devices, to receive data from the one or more external devices, and to send data to the one or more external devices. Such external devices along with the various internal devices may also be known as peripheral devices. The I/O interface may include both electrical and physical connections for operably coupling the various peripheral devices to the computing machine or the processor. The I/O interface may be configured to communicate data, addresses, and control signals between the peripheral devices, the computing machine, or the processor. The I/O interface may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface may be configured to implement only one interface or bus technology.

Alternatively, the I/O interface may be configured to implement multiple interfaces or bus technologies. The I/O interface may be configured as part of, all of, or to operate in conjunction with, the system bus. The I/O interface may include one or more buffers for buffering transmissions between one or more external devices, internal devices, the computing machine, or the processor.

The I/O interface may couple the computing machine to various input devices including mice, touch-screens, scanners, electronic digitizers, sensors, receivers, touchpads, trackballs, cameras, microphones, keyboards, any other pointing devices, or any combinations thereof. The I/O interface may couple the computing machine to various output devices including video displays, speakers, printers, projectors, tactile feedback devices, automation control, robotic components, actuators, motors, fans, solenoids, valves, pumps, transmitters, signal emitters, lights, and so forth.

The computing machine may operate in a networked environment using logical connections through the network interface to one or more other systems or computing machines across the network. The network may include wide area networks (WAN), local area networks (LAN), intranets, the Internet, wireless access networks, wired networks, mobile networks, telephone networks, optical networks, or combinations thereof. The network may be packet switched, circuit switched, of any topology, and may use any communication protocol. Communication links within the network may involve various digital or an analog communication media such as fiber optic cables, free-space optics, waveguides, electrical conductors, wireless links, antennas, radio-frequency communications, and so forth. The processor may be connected to the other elements of the computing machine or the various peripherals discussed herein through the system bus. It should be appreciated that the system bus may be within the processor, outside the processor, or both. According to some embodiments, any of the processor, the other elements of the computing machine, or the various peripherals discussed herein may be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

Reagents, tools, and/or instructions for performing the methods described herein can be provided in a kit. Such a kit can include reagents for collecting a tissue sample from a patient, such as by biopsy, and reagents for processing the tissue. The kit can also include one or more reagents for performing a expression level analysis, such as reagents for performing nucleic acid amplification, including RT-PCR and qPCR, NGS, northern blot, proteomic analysis, or immunohistochemistry to determine expression levels of LINGO-1, or of KCNN1 or CDH23, in a sample of a patient. For example, primers for performing RT-PCR, probes for performing northern blot analyses, and/or antibodies or aptamers, as discussed herein, for performing proteomic analysis such as Western blot, immunohistochemistry and ELISA analyses can be included in such kits. Appropriate buffers for the assays can also be included. Detection reagents required for any of these assays can also be included. The kits may be array or PCR based kits for example and may include additional reagents, such as a polymerase and/or dNTPs for example. The kits featured herein can also include an instruction sheet describing how to perform the assays for measuring expression levels. The kit may include one or more primer pairs complementary to LINGO-1, or to KCNN1 or CDH23. The kit may also include one or more primers pairs complementary to a reference gene.

Kits for detecting and/or diagnosing sarcoma in a subject may permit the methylation status of LINGO-1, or of KCNN1 or CDH23, to be determined. Such kits may include primers and/or probes for determining the methylation status of the gene directly. They may thus comprise methylation specific primers and/or probes that discriminate between methylated and unmethylated forms of DNA by hybridization. Such kits will typically also contain a reagent that selectively modifies either the methylated or non-methylated form of CpG dinucleotide motifs. Suitable chemical reagents comprise hydrazine and bisulphite ions. An example is sodium bisulphite. The kits may, however, contain other reagents as discussed hereinabove to determine methylation status such as restriction endonucleases.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing a gene expression analysis and interpreting the results.

The kit may further comprise a computer application or storage medium as described above.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the examples described herein.

Although specific embodiments have been described above in detail, the description is merely for purposes of illustration. It should be appreciated, therefore, that many aspects described above are not intended as required or essential elements unless explicitly stated otherwise.

Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

### DESCRIPTION OF THE FIGURES

FIG. 1: Analysis of LINGO-1 expression levels in Ewing's Sarcoma (EWS) cell lines compared to mesenchymal stem cells (MSCs) by quantitative PCR (qPCR).

RNA from seven EWS cell lines as well as three MSC lines was isolated, reverse transcribed into cDNA and analysed by qPCR. LINGO-1 expression levels were normalized against the house-keeping gene GAPDH and the EWS cell line A673 was used as a reference. Relative expression levels are given as 2^{-ΔΔCt} with ΔCt=Ct_{LINGO1}−Ct_{GAPDH}, ΔΔCt=ΔCt _{Sample}−Ct_{A673}.

FIG. 2: Western blot analysis of LINGO-1 expression.

Cells were lysed in RIPA lysis buffera and protein concentrations were measured using the BCA assay (Thermo Scientific). Subsequently, the lysates were separated by SDS gel electrophoresis and analysed by western blotting with the anti-LINGO-1 antibody (Abcam) and the anti-beta-Actin antibody (Sigma).

FIG. 3: Analysis of LINGO1 expression levels in EWS cell lines compared to MSCs by quantitative PCR (qPCR).

FIG. 4: Analysis of the expression levels of KCNN1 (FIG. 4A) and CDH23 (FIG. 4B) in Ewing's Sarcoma (EWS) cell lines compared to mesenchymal stem cells (MSCs) by quantitative PCR (qPCR).

FIG. 5: Analysis of LINGO1 expression levels in Ewing's Sarcoma patient samples.

FIG. 6: ADCC assay targeting LINGO1 overexpressing EWS cell line A673_lingo151 and MSC5Blast cells

FIG. 7: ADCC assay targeting CHO cells expressing LINGO1

### EXAMPLES

The present invention will be further understood by reference to the following experimental examples.

### Example 1

### Results & Discussion

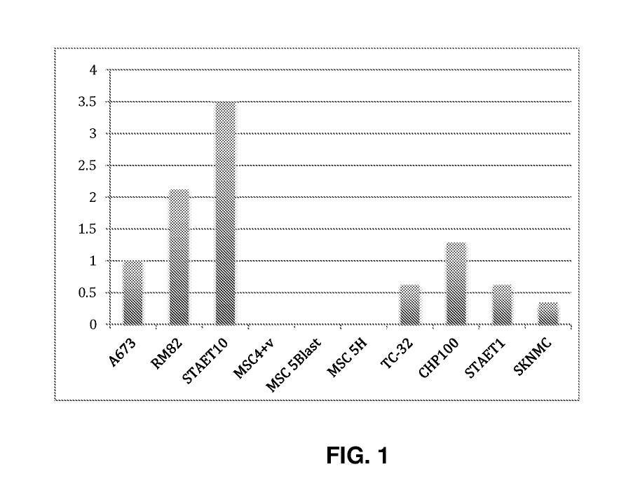

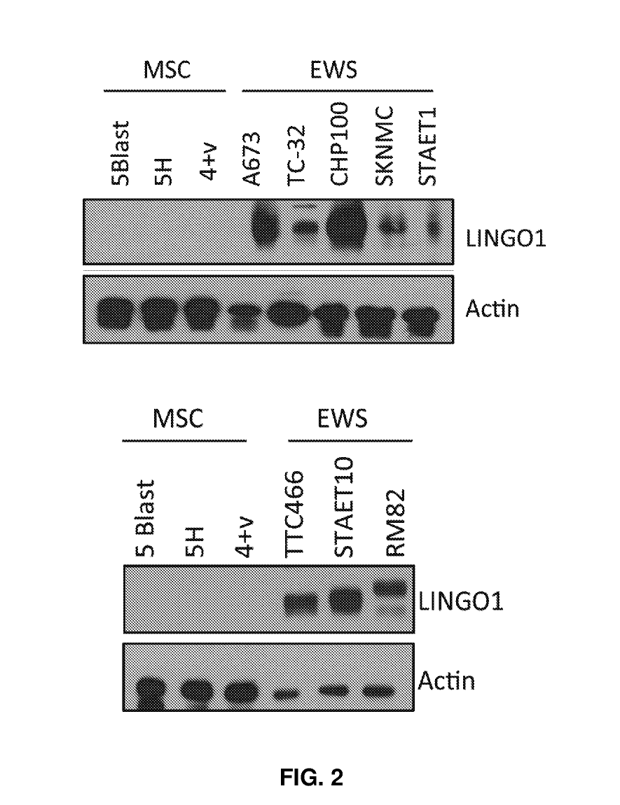

Recently, Next Generation Deep sequencing has been applied to RNA sequencing (RNA-seq) and this has emerged as a new method for the comprehensive analysis of the whole cellular transcriptomes and is a powerful surrogate tool for the analysis of the cell surface proteins (herein termed the surfaceome)^{6,7}. Cellular RNA is converted into a library of cDNA, which is then sequenced in a high-throughput manner. We have carried out RNA-seq deep sequencing on three different human Ewing's Sarcomas (EWS) and compared these data with two mesenchymal stem cell (MSC) lines (MSC is thought to be the cell of origin of Ewing's sarcoma). Using an improved version of the original surfaceome database⁶, we have found several mRNAs that encode for cell surface proteins that are un-regulated in EWS compared with the MSCs. One of these encodes LINGO-1. RNA-seq RPKM values shown in Table 1 show that LINGO-1 mRNA is more than 2500-fold higher in level in the three EWS cells compared with the MSC. Quantitative PCR (qPCR) (FIG. 1, Table 2) shows that LINGO1 mRNA is highly expressed in seven EWS lines but is negligible in three MSC cell lines. Examination of LINGO-1 protein expression by Western blotting (FIG. 2) confirmed the RNA findings and showed a direct correlation to mRNA level demonstrating that LINGO-1 protein is restricted to EWS tumours when compared to the normal counterpart. In healthy individuals, LINGO1 is exclusively expressed in the central nervous system where it plays a role in the regulation of neuronal survival, axon regeneration, oligodendrocyte differentiation and myelination⁸⁻¹⁰. LINGO1 consists of 620 aa with a large extracellular domain (42-561) the structure of which has been elucidated by crystallography¹¹. Its well-defined and accessible extracellular domain, as well as its specificity for Ewing's sarcoma makes LINGO1 a biomarker for diagnosis, for residual disease detection and a therapeutic target of Ewing's sarcoma with methods that target the expressed protein or the mRNA or target the gene or target the molecules that control the expression of LINGO-1 in EWS or functional properties of LINGO-1.

TABLE 1
Table 1: RPKM values for key surfaceome mRNAs

| gene | MSC 4 + V | MSC 5H | TTC-466 | TC-32 | A673 |
|---|---|---|---|---|---|
| LINGO1 | 0.066061371 | 0 | 97.60931409 | 107.2860281 | 51.55255273 |

The MSC lines 4+v and 5H as well as the three EWS cell lines TTC-466, TC-32 and A673 were analysed by RNA-seq.

TABLE 2
Table 2: Ct values of qPCR experiments analysing LINGO1 expression.

| Sample | Ct value |
|---|---|
| A673 | 24.48439217 |
| RM82 | 24.82822704 |
| STAET10 | 26.1312542 |
| TC-32 | 24.47195435 |
| CHP100 | 24.27594185 |
| STAET1 | 22.27841377 |
| SKNMC | 23.84445572 |
| MSC 4 + v | 35.33303452 |
| MSC 5Blast | 33.84421539 |
| MSC 5H | 35.58719635 |

RNA from seven EWS cell lines as well as three MSC lines was isolated, reverse transcribed into cDNA and analysed by qPCR.

### Example 2

### Analysis of LINGO1 Expression Levels in EWS Cell Lines Compared to MSCs by Quantitative PCR (qPCR)

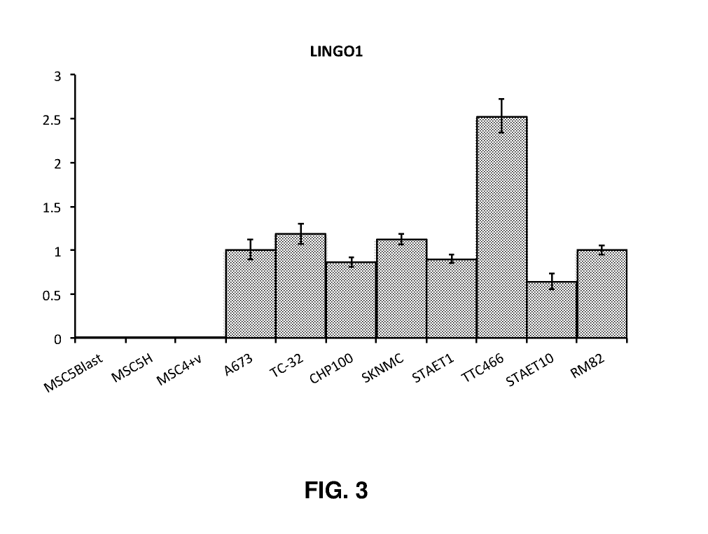

RNA from seven EWS cell lines as well as three MSC lines was isolated, reverse transcribed into cDNA and analysed by qPCR. LINGO1 expression levels were normalized against the house-keeping gene GAPDH and the EWS cell line A673 was used as a reference. The results are shown in FIG. 3. Relative expression levels are given as RQ (relative quantification)=2^{-ΔΔCt} with ΔCt=Ct_{LINGO1}−Ct_{GAPDH}, ΔΔCt=ΔCt Sample−ΔCt_{A673}. The error bars represent the 95% confidence interval of the RQ value. It is readily seen that LINGO1 is expressed in all EWS cell lines but there is no expression in MSC lines.

### Example 3

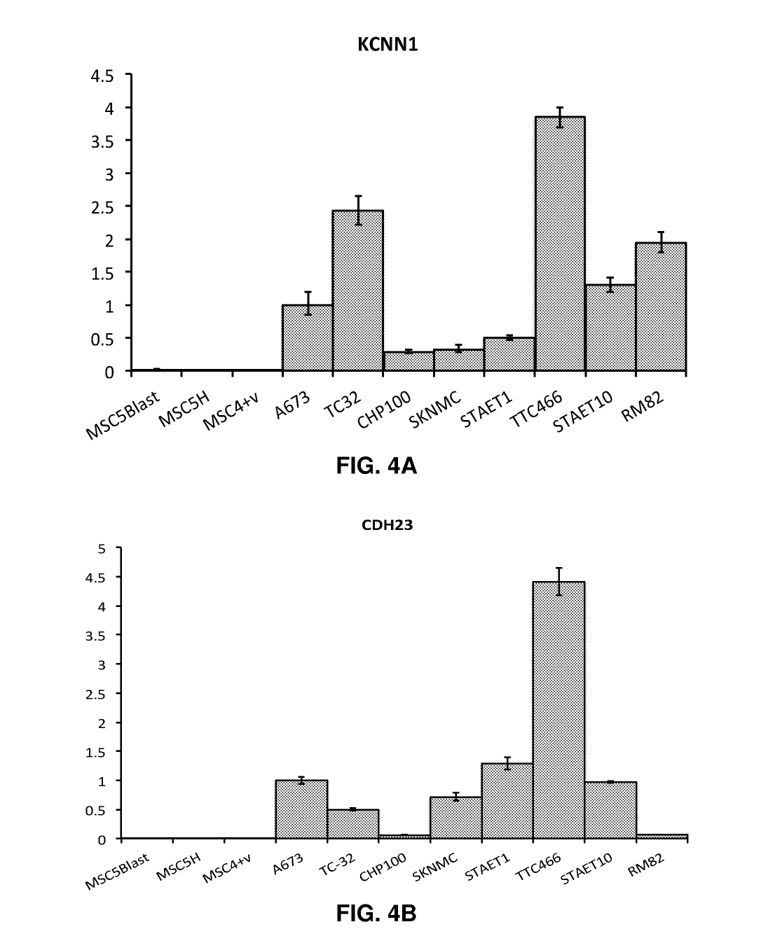

### Analysis of the Expression Levels of KCNN1 (FIG. 4A) and CDH23 (FIG. 4B) in Ewing's Sarcoma (EWS) Cell Lines Compared to Mesenchymal Stem Cells (MSCs) by Quantitative PCR (qPCR)

RNA from seven EWS cell lines as well as three MSC lines was isolated, reverse transcribed into cDNA and analysed by qPCR. Expression levels were normalized against the house-keeping gene GAPDH and the EWS cell line A673 was used as a reference. Results are shown in FIG. 4. Relative expression levels are given as RQ (relative quantification)=2−^{ΔΔCt} with ΔCt=Ct_{KCNN1/CDH23}−Ct_{GAPDH}, ΔΔCt=ΔCt_{Sample}−ΔCt_{A673}. The error bars represent the 95% confidence interval of the RQ value. It is readily seen from FIG. 4A that KCNN1 is expressed in all EWS cell lines but there is no expression in MSC lines. Similarly, it is readily seen from FIG. 4B that CDH23 is expressed in all EWS cell lines but there is no expression in MSC lines.

### Example 4

### Analysis of LINGO1 Expression Levels in Ewing's Sarcoma Patient Samples

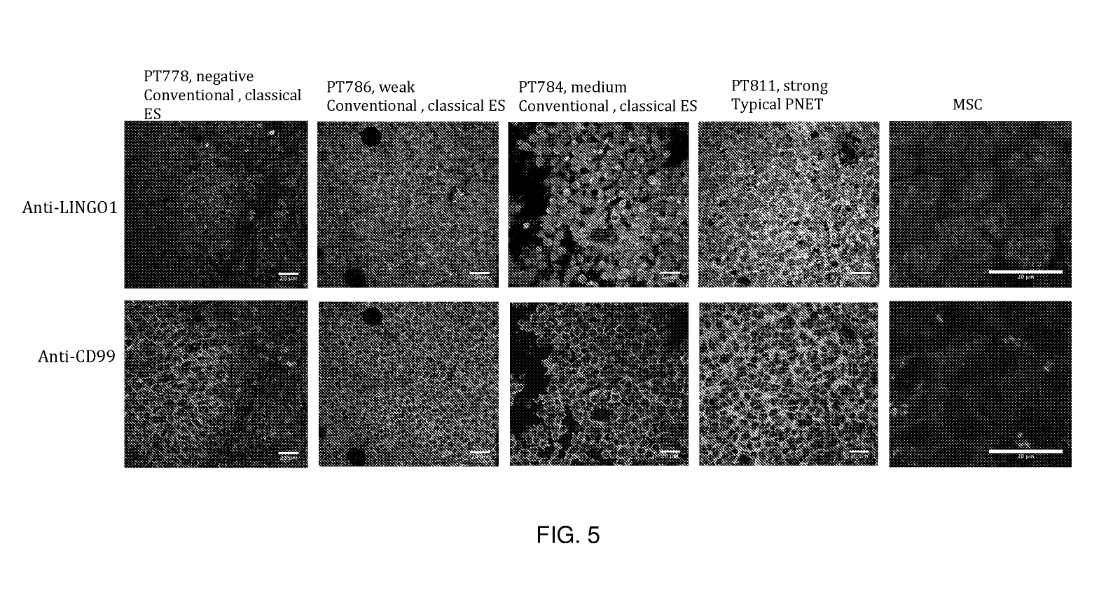

Tissue microarrays containing cores from tumour biopsies were analysed by immunohistochemistry using a mixture of two anti-LINGO1 antibodies (Abcam and Milipore) and anti-CD99 antibody (Thermo Scientific) together with AlexaFluor 488-coupled (LINGO1) and Alexa-Fluor594-coupled secondary antibodies (Invitrogen). Control spreads of MSC cells were used as a control. Images were acquired by confocal laser scanning microscopy and analysed using ImageJ software. A total of 56 cases were analysed. 5 cases (8.9%) were negative for LINGO1, 12 cases (21.4%) showed weak staining, 25 (44.6%) showed moderate staining and 14 (25%) showed strong staining for LINGO1. Representative results are shown in FIG. 5. Scale bars=20 μm.

### Example 5

### ADCC Assay Targeting LINGO1 Overexpressinq EWS Cell Line A673 Lingo151 and MSC5Blast Cells

An ADCC Reporter Bioassay (Promega) was performed in order to confirm targeting of LINGO1 overexpressing Ewing's Sarcoma (EWS) cells.

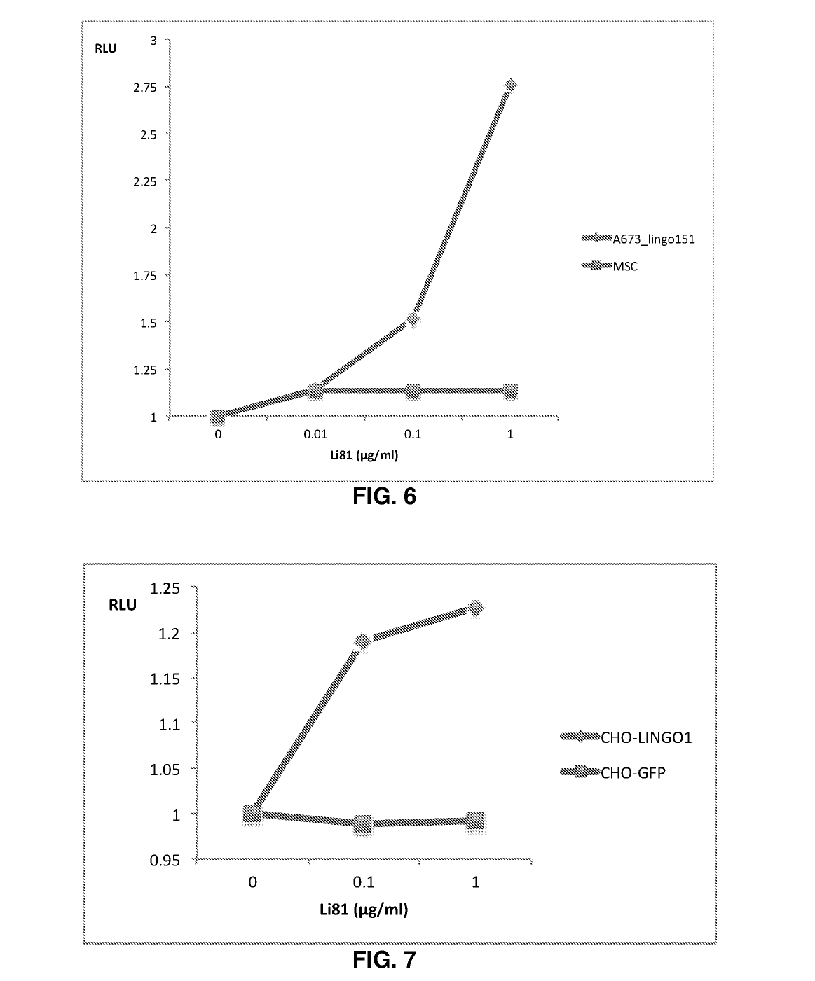

5×10⁵ target cells per well were seeded in a 24-well plate. The following day the medium was exchanged to serum-free medium and the anti-LINGO1 antibody Li81 was added at the indicated concentrations (x axis in FIG. 6). Jurkat Bioeffector cells (Promega) stably expressing the CD16 (FcγRIIIa) receptor and an NFAT response element driving expression of firefly luciferase were used as effector cells to detect ADCC (per the Promega protocol). 1.2×10⁶ effector cells per well were added to the target cells and incubated for 6 hours at 37° C. Luciferase activity in the effector cells (y axis in FIG. 6) is a measure of activation of the NFAT pathway in the effector cells and is used as a read-out for ADCC activity. Luciferase activity was quantified using a plate reader and is given as RLU (relative light units) normalized to background. Representative results are shown in FIG. 6 and confirm ADCC pathway activation caused by the anti-LINGO1 antibody targeting the EWS cells (and not the MSCs, which do not express LINGO1).

### Example 6

### ADCC Assay Targeting CHO Cells Expressing LINGO1

An ADCC Reporter Bioassay (Promega) was performed in order to confirm targeting of LINGO1 overexpressing CHO cells.

CHO cells were seeded in 24-well plates and transiently transfected with LINGO1 or GFP expression vectors. 24 hours after transfection, ADCC assays (Promega) were conducted. The LINGO1-specific antibody Li81 was added at the indicated concentrations (x-axis in FIG. 7) and Jurkat Bioeffector cells (Promega) stably expressing the CD16 (FcγRIIIa) receptor and an NFAT response element driving expression of firefly luciferase were used as effector cells. 1.2×10⁶ effector cells per well were added to the target cells and incubated for 6 hours at 37° C. Luciferase activity in the effector cells is a measure of activation of the NFAT pathway in the effector cells and is used as a read-out for ADCC activity (y-axis in FIG. 7). Luciferase activity was quantified using a plate reader and is given as RLU (relative light units) normalized to background. Representative results are shown in FIG. 7 and confirm ADCC pathway activation caused by the anti-LINGO1 antibody targeting the CHO cells transiently expressing LINGO1 (and not the CHO cells transfected with GFP).

### REFERENCES

1 Sankar, S. & Lessnick, S. L. Promiscuous partnerships in Ewing's sarcoma. Cancer genetics 204, 351-365, doi:10.1016/j.cancergen.2011.07.008 (2011).
2 Terrier, P., Llombart-Bosch, A. & Contesso, G. Small round blue cell tumors in bone: prognostic factors correlated to Ewing's sarcoma and neuroectodermal tumors. Seminars in diagnostic pathology 13, 250-257 (1996).
3 Abed, R. & Grimer, R. Surgical modalities in the treatment of bone sarcoma in children. Cancer treatment reviews 36, 342-347, doi:10.1016/j.ctrv.2010.02.010 (2010).
4 Potratz, J., Dirksen, U., Jurgens, H. & Craft, A. Ewing sarcoma: clinical state-of-the-art. Pediatric hematology and oncology 29, 1-11, doi:10.3109/08880018.2011.622034 (2012).
5 Gorlick, R., Janeway, K., Lessnick, S., Randall, R. L. & Marina, N. Children's Oncology Group's 2013 blueprint for research: bone tumors. Pediatric blood & cancer 60, 1009-1015, doi:10.1002/pbc.24429 (2013).
6 da Cunha, J. P. et al. Bioinformatics construction of the human cell surfaceome. Proceedings of the National Academy of Sciences of the United States of America 106, 16752-16757, doi:10.1073/pnas.0907939106 (2009).
7 Wang, Z., Gerstein, M. & Snyder, M. RNA-Seq: a revolutionary tool for transcriptomics. Nature reviews. Genetics 10, 57-63, doi:10.1038/nrg2484 (2009).
8 Mi, S., Sandrock, A. & Miller, R. H. LINGO-1 and its role in CNS repair. The international journal of biochemistry & cell biology 40, 1971-1978, doi:10.1016/j.biocel.2008.03.018 (2008).
9 Mi, S. et al. LINGO-1 negatively regulates myelination by oligodendrocytes. Nature neuroscience 8, 745-751, doi:10.1038/nn 1460 (2005).
10 Llorens, F. et al. Developmental analysis of Lingo-1/Lern1 protein expression in the mouse brain: interaction of its intracellular domain with Myt1I. Developmental neurobiology 68, 521-541, doi:10.1002/dneu.20607 (2008).
11 Mosyak, L. et al. The structure of the Lingo-1 ectodomain, a module implicated in central nervous system repair inhibition. The Journal of biological chemistry 281, 36378-36390, doi:10.1074/jbc.M607314200 (2006).

The invention may be further defined in the following set of numbered clauses:
1. A specific binding agent that can specifically bind to LINGO-1 polypeptide, or to KCNN1 or CDH23 polypeptide, and elicit an anti-tumour response.
2. The specific binding agent of clause 1 wherein the specific binding agent comprises an antibody or an aptamer.
3. The specific binding agent of clause 2 wherein the anti-tumour response is Antibody-Dependent Cell-Mediated Cytotoxicity (ADCC) or Aptamer-Dependent Cell-Mediated Cytotoxicity.
4. A specific binding agent that can specifically bind to LINGO-1 polypeptide, or to KCNN1 or CDH23 polypeptide, wherein the specific binding agent is operably connected to:
   i. a cytotoxic agent or prodrug thereof,
   ii. an agent capable of activating a prodrug that when activated is a cytotoxic agent; or
   iii. a delivery agent containing a cytotoxic agent or prodrug thereof.
5. The specific binding agent of clause 4 wherein the specific binding agent comprises an antibody or an aptamer.
6. The specific binding agent of clause 4 or 5 wherein the agent capable of activating a prodrug is an enzyme.
7. The specific binding agent of clause 4 or 5 wherein the delivery agent is a liposome.
8. The specific binding agent of any of clauses 4 to 7 wherein the cytotoxic agent is a chemotherapeutic drug or a RNA expression vector.
9. The specific binding agent of clause 8 wherein the RNA expression vector encodes an shRNA and/or an siRNA and/or an antisense RNA.
10. The specific binding agent of clause 9 wherein the shRNA and/or siRNA and/or antisense RNA is capable of reducing expression of the EWS-FLI1 fusion protein.
11. The specific binding agent of clause 8 wherein the RNA expression vector encodes an antibody or aptamer capable of inhibiting the activity of the EWS-FLI1 fusion protein.
12. The specific binding agent of any of clauses 2, 3, or 5-11 wherein the aptamer that can specifically bind to LINGO-1 polypeptide, or to KCNN1 or CDH23 polypeptide, and/or the aptamer capable of reducing expression of the EWS-FLI1 fusion protein is a peptide aptamer or an RNA aptamer.
13. A pharmaceutical composition comprising a specific binding agent according to any one of clauses 1 to 12 and a pharmaceutically acceptable carrier or excipient.
14. A kit for targeting LINGO-1 expressing cells, or for targeting KCNN1 or CDH23 expressing cells, comprising:
   a. a specific binding agent that can specifically bind to LINGO-1 polypeptide, or to KCNN1 or CDH23 polypeptide; and
   b. one or more of:
      i. a cytotoxic agent or prodrug thereof,
      ii. an agent capable of activating a prodrug that when activated is a cytotoxic agent;or
      iii. a delivery agent containing a cytotoxic agent or prodrug thereof.
15. The kit of clause 14, wherein the specific binding agent is an antibody or an aptamer.
16. The kit of clause 14 or 15 further comprising means for connecting components a and b.
17. A specific binding agent as described in any of clauses 1 to 12 or pharmaceutical composition as described in clause 13 for use as a medicament.
18. A specific binding agent as described in any of clauses 1 to 12 or pharmaceutical composition as described in clause 13 for use in a method of treating sarcoma.
19. A specific binding agent that can specifically bind to LINGO-1 polypeptide, or to KCNN1 or CDH23 polypeptide, for use in a method of treating sarcoma.
20. The specific binding agent for use of clause 19 wherein the specific binding agent is an antibody or an aptamer.
21. A method for treating sarcoma in a subject comprising inhibiting LINGO-1 expression or LINGO-1 protein activity, or inhibiting KCNN1 or CDH23 expression or protein activity.
22. The method of clause 21 comprising administering to a subject a specific binding agent, preferably an antibody or aptamer, that can specifically bind to LINGO-1 polypeptide, or to KCNN1 or CDH23 polypeptide.
23. A method for treating sarcoma in a subject comprising administering to a subject the specific binding agent of any of clauses 1 to 12 or pharmaceutical composition of clause 13.
24. The method of clause 22 or 23 wherein the specific binding agent or pharmaceutical composition is administered systemically.
25. The specific binding agent of any of clauses 1 to 12, the pharmaceutical composition of clause 13, the kit of clauses 14 to16, the specific binding agent for use of any of clauses 17 to 20 or the method of any of clauses 21 to 24 wherein the specific binding agent cannot cross the blood brain barrier.
26. A method for detecting and/or diagnosing sarcoma in a subject comprising:
determining the expression level of LINGO-1 in a sample from the subject, or determining the expression level of KCNN1 or CDH23 in a sample from the subject, wherein the determined expression level is used to detect and/or diagnose sarcoma in the subject.
27. The method of any of clauses 21 to 26 or the specific binding agent for use of any of clauses 17 to 20 wherein the sarcoma is a sarcoma that expresses the EWS-FLI1 fusion protein.
28. The method any of clauses 21 to 26 or the specific binding agent for use of any of clauses 17 to 20 wherein the sarcoma is Ewing's sarcoma, neuroblastoma and/or rhabdomyosarcoma.
29. The method of any of clauses 21 to 26 or the specific binding agent for use of any of clauses 17 to 20 wherein the sarcoma is Ewing's sarcoma.
30. The method of any of clauses 26 to 29 comprising comparing the expression level to a reference value or to one or more control samples.
31. The method of any of clauses 26 to 30 wherein the expression level is determined at the level of protein or RNA.
32. The method of clause 31 wherein the expression level is determined by immunohistochemistry or western blot.
33. The method of clause 31 wherein the expression level is determined by quantitative PCR.
34. The method of any of clauses 26 to 33 further comprising obtaining the sample from the subject.
35. The method of any of clauses 26 to 34 wherein the sample comprises, consists essentially of or consists of bone and/or soft tissue.
36. The method of any of clauses 26 to 35 wherein detecting comprises detecting residual disease following treatment.
37. Use of an antibody that binds specifically to LINGO-1, or an antibody that binds specifically to KCNN1 or CDH23, for detecting and/or diagnosing sarcoma in a subject.
38. The use of clause 37 wherein the antibody is conjugated to a label.
39. The use of clause 38 wherein the label is a fluorophore.
40. A method for detecting and/or diagnosing sarcoma in a subject comprising:
determining the expression level of LINGO-1 in a sample from the subject, or determining the expression level of KCNN1 or CDH23 in a sample from the subject, in order to identify the presence or absence of cells characteristic of sarcoma wherein the determined presence or absence of the cells is used to detect and/or diagnose sarcoma in the subject.
41. A method for detecting metastases in a subject with sarcoma comprising:
determining the expression level of LINGO-1 in a sample from the subject, or determining the expression level of KCNN1 or CDH23 in a sample from the subject, wherein the determined expression level is used to detect metastases.
42. The method of clause 41 wherein the sample is a body fluid, optionally wherein the sample is peripheral blood.
43. A method for identifying residual disease in a subject following treatment for sarcoma comprising:
determining the expression level of LINGO-1 in a sample from the subject wherein the determined expression level is used to identify residual disease in the subject.
44. A method for detecting and/or diagnosing sarcoma in a subject comprising:
   a. obtaining a sample from the subject
   b. applying a specific binding agent that can specifically bind to LINGO-1 polypeptide, or to KCNN1 or CDH23 polypeptide, to the sample from the subject
   c. applying a detection agent that detects the specific binding agent-LINGO-1 polypeptide complex, or to the specific binding agent-KCNN1 or specific binding agent-CDH23 polypeptide complex,
   d. using the detection agent to determine the level of LINGO-1 polypeptide, or the level of KCNN1 or CDH23 polypeptide,
wherein the determined level of the LINGO-1 polypeptide, or the level of KCNN1 or CDH23 polypeptide,
is used to detect and/or diagnose sarcoma in the subject.
45. The method of clause 44 wherein the specific binding agent is an antibody or an aptamer.
46. A system or device for performing the method of any previous clause.
47. A system or test kit for detecting and/or diagnosing sarcoma in a subject comprising:
   a. one or more testing devices for determining the expression level of LINGO-1, or of KCNN1 or CDH23, in a sample from the subject
   b. a processor; and
   c. a storage medium comprising a computer application that, when executed by the processor, is configured to:
      i. access and/or calculate the determined expression level of LINGO-1, or of KCNN1 or CDH23, in the sample on the one or more testing devices
      ii. calculate whether there is an increased or decreased level of LINGO-1, or of KCNN1 or CDH23, in the sample; and
      iii. output from the processor the detection and/or diagnosis of the sarcoma.
48. The system or test kit of clause 47 further comprising a display for the output from the processor.
49. A computer application or storage medium comprising a computer application as defined in clause 47 or 48.
50. A chimeric antigen receptor (CAR) comprising:
   a. a specific binding agent that can specifically bind to LINGO-1 polypeptide, or to KCNN1 or CDH23 polypeptide,
   b. a hinge domain
   c. a trans-membrane domain
   d. an endodomain which transmits signals within a T cell comprising the CAR.
51. A T cell comprising a CAR as defined in clause 50.
52. A nucleic acid molecule encoding a CAR as defined in clause 50.
53. A pharmaceutical composition comprising a T cell according to clause 51 and a carrier or excipient.
54. A method of treating sarcoma comprising administering to a subject a T cell as defined in clause 51 or a pharmaceutical composition as defined in clause 52.
55. The method according to clause 53 which is an autologous T cell therapy.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1-28. (canceled)

29. A method of treating sarcoma in a subject in need thereof, the method comprising administering to the subject a specific binding agent that can specifically bind to LINGO-1, KCNN1 or CDH23 polypeptide.

30. The method of claim 29, wherein the specific binding agent is an antibody or an aptamer.

31. The method of claim 29, wherein the specific binding agent mediates Antibody-Dependent Cell-Mediated Cytotoxicity (ADCC) or Aptamer-Dependent Cell-Mediated Cytotoxicity.

32. The method of claim 29, wherein the specific binding agent is operably connected to:
a. a cytotoxic agent or prodrug thereof,
b. an agent capable of activating a prodrug that when activated is a cytotoxic agent; or
c. a delivery agent containing a cytotoxic agent or prodrug thereof.

33. The method of claim 32, wherein the agent capable of activating a prodrug is an enzyme.

34. The method of claim 32, wherein the delivery agent is a liposome.

35. The method of claim 32, wherein the cytotoxic agent is a chemotherapeutic drug or a RNA expression vector.

36. The method of claim 35, wherein the RNA expression vector encodes a shRNA and/or an siRNA and/or an antisense RNA.

37. The method of claim 36, wherein the shRNA and/or siRNA and/or antisense RNA is capable of reducing expression of the EWS-FLI1 fusion protein.

38. The method of claim 35, wherein the RNA expression vector encodes an antibody or aptamer capable of inhibiting the activity of the EWS-FLI1 fusion protein.

39. The method of claim 30, wherein the aptamer that can specifically bind to LINGO-1, KCNN1 or CDH23 polypeptide is a peptide aptamer or an RNA aptamer.

40. The method of claim 29, wherein the specific binding agent is administered systemically and the specific binding agent cannot cross the blood brain barrier.

41. The method of claim 29, wherein the sarcoma expresses the EWS-FLI1 fusion protein.

42. The method of claim 29, wherein the sarcoma is Ewing's sarcoma, neuroblastoma and/or rhabdomyosarcoma.

43. The method of claim 29, comprising administering the specific binding agent as a chimeric antigen receptor (CAR) comprising:
a. a specific binding agent that can specifically bind to LINGO-1, KCNN1 or CDH23 polypeptide;
b. a hinge domain;
c. a trans-membrane domain; and
d. an endodomain which transmits signals within a T cell comprising the CAR; or a nucleic acid molecule encoding the CAR.

44. The method of claim 43, comprising administering the CAR as a T cell comprising the CAR.

45. A chimeric antigen receptor (CAR) comprising:
a. a specific binding agent that can specifically bind to LINGO-1, KCNN1 or CDH23 polypeptide;
b. a hinge domain;
c. a trans-membrane domain; and
d. an endodomain which transmits signals within a T cell comprising the CAR; or a nucleic acid molecule encoding the CAR

46. A T cell comprising the CAR of claim 45.

47. A pharmaceutical composition comprising:
(a) the T cell of claim 46; and
(b) a carrier or excipient.

48. A method comprising detecting the expression level of LINGO-1, KCNN1 or CDH23 in a biological sample from a patient.


SEQUENCE LISTING (1 sequence)

SEQ ID NO: 1 - DNA, 71 bp, Artificial sequence
  shRNA sequence within pSRP-EF3′-2 vector
gatccccatagaggtgggaagcttatttcaagagaataagcttcccacctctattttttggaactcgaggt